(19) United States
(12) Patent Application Publication
(10) Pub. No.: US 2007/0043391 A1
(43) Pub. Date: Feb. 22, 2007

(54) Occlusion device and method of for its production

(75) Inventors: Robert Moszner, Bad Klosterlausnitz (DE); Hans Reiner Figulla, Jena (DE); Friedrich Moszner, Hohlstedt (DE); Rudiger Ottma, Grossschwabhausen (DE)

Correspondence Address: AKERMAN SENTERFITT, 801 PENNSYLVANIA AVENUE N.W., SUITE 600, WASHINGTON, DC, 20004, US

(73) Assignee: JEN.MEDITECH GMBH, Jena (DE)

(21) Appl. No.: 10/551,687

(22) PCT Filed: Aug. 18, 2004
(86) PCT No.: PCT/EP04/09279

§ 371 Date: Aug. 7, 2006

(30) Foreign Application Priority Data
Aug. 22, 2003 (DE) 103 38 702.1

(51) Int. Cl. A61B 17/08

(52) U.S. Cl. 606/213

(57) ABSTRACT

An occlusion device and manufacturing includes of thin wires or threads made from a shape-memory material. In the expanded state, the occlusion device exhibits a proximal and a distal retention area as well as a cylindrical crosspiece interposed therebetween. Because the proximal retention area of braiding exhibits a form which flares toward the proximal end, in the inserted state of the occlusion device, the lateral edge of proximal retention area lies substantially flush with the septum wall and retention area does not protrude past the septum wall. The manufacturing method includes a braiding technique for producing a tubular braiding open to the top and which needs only be provided with a holder for bundling the threads or wires of the braiding at one end, while at the opposite side, the threads or wires of braiding are intertwined from the center thereof.

The present invention relates to an occlusion device consisting of a braiding of thin wires or threads given a suitable form by means of a molding and heat treatment procedure, having a proximal and a distal retention area, whereby the ends of the wires or threads converge into a holder in the distal retention area, and having a cylindrical crosspiece interposed between said proximal and distal retention areas, whereby the two retention areas are usually positioned on the two sides of a shunt to be occluded in a septum by means of an intravascular surgical procedure while the crosspiece transverses the shunt. The invention moreover relates to a method for producing said occlusion device.

Medical technology has long endeavored to be able to occlude septal defects, for instance atrioseptal defects, with non-surgical transvenous catheter intervention, in other words, without having to perform an, operation in the literal sense. Various different occlusion systems have been proposed each with their own pros and cons, without any one specific occlusion system having yet become widely accepted. In making reference to these different systems, the following will use the terms “occluder” or “occlusion device.”

In all interventional occlusion systems, a self-expanding umbrella system is introduced transvenously into a defect to be occluded in a septum. This type of system might comprise two umbrellas; one, for example, positioned at the distal side of the septum (i.e. the side furthest from the median plane of the body/heart) and one at the proximal side of the septum (i.e. the side closer to the median plane of the body), whereby the two umbrella prostheses are subsequently secured to a double umbrella in the septal defect. Thus, in the assembled state, the occlusion system usually consists of two braced umbrellas connected to one another by means of a short bolt transversing the defect. However, a disadvantage to such prior art occlusion devices turns out to be the relatively complicated, difficult and complex implantation procedure. Apart from the complicated implantation of the occlusion system in the septal defect to be occluded, the umbrellas utilized are susceptible to material fatigue along with fragment fracture. Furthermore, thromboembolic complications are frequently to be anticipated.

In order to enable the inventive occlusion device to be introduced by means of a surgical insertion instrument and/or guidewire, a holder is provided at the end of the distal retention area which can engage with the insertion instrument and/or guidewire. It is thereby intended that this engagement can be readily disengaged after positioning the occlusion device in the defect. For example, it is possible to devise the braiding at the end of the distal retention area of the occlusion device in such a manner so as to create an internal threading in the holder to engage with the insertion instrument. Of course, other embodiments are naturally also conceivable.

With another type of occlusion device, the so-called Lock-Clamshell umbrella system, two stainless steel preferably Dacron-covered umbrellas are provided, each stabilized by four arms. This type of occluder is implanted into the patient through a vein. However, seen as problematic with the Lock-Clamshell occluder is the fact that the insertion instruments necessary to implant the device need to be of relatively large size. A further disadvantage is that many different occluder sizes are needed in order to cope with the respective proportions of the septal-defects to be occluded. It thus turns out that the umbrellas do not flatten out completely in the inserted state if the length or the diameter of the crosspiece inserted into the defect is not of an optimum match. This results in incomplete endothelialization. It has furthermore been shown that many of the systems implanted into patients' bodies exhibit material fatigue and fractures in the metallic structures due to the substantial mechanical stresses over a longer period. This is especially the case given permanent stress between an implant and the septum.

In order to overcome these disadvantages, self-centering occlusion devices have been developed which are inserted into the body of the patient and introduced into the septal defect to be occluded by way of a minimally invasive procedure, for example using a catheter and guidewires. Their design is based on the principle that the occlusion device can be tapered to the dimensions of the insertion instrument and/or catheter used for the intravascular surgical procedure. Such a tapered occlusion device is then introduced by catheter into the septal defect to be occluded, respectively into the shunt to be occluded of the septum defect. The occluder is then discharged from the catheter, upon which the self-expanding umbrellas, retention discs respectively, subsequently unfold against the two sides of the septum. The umbrellas in turn comprise fabric inserts manufactured from or covered by, for example, Dacron, with which the defect/shunt is occluded. The implants remaining in the body are more or less completely ingrown by the body's own tissue after a few weeks or months.

An example of a self-centering occlusion device of the type specified at the outset and in accordance with the pre-characterizing part of claim 1 is known from WO 99/12478 A1, which is a further refinement of the occlusion device known as the “Amplatzer-occluder” in accordance with printed U.S. Pat. No. 5,725,552, a braiding of a plurality of fine, intertwined nitinol wire strands in the shape of a yo-yo. Each braiding is manufactured in its original form as a rounded braiding having loose wire ends both at its leading end (its proximal side, respectively) as well as at its trailing end (its distal side, respectively). During the subsequent processing of the rounded braiding, each of these loose ends must then be gathered into a sleeve and welded together. After the appropriate processing, both the proximal side as well as the distal side of the finished occluder exhibit a protruding collar. Dacron patches are sewn into the distal and proximal retention umbrellas and the interposed crosspiece. Because of the memory effect exhibited by the nitinol material used, the two retention umbrellas unfold by themselves upon exiting the catheter, initially in a balloon-like intermediate stage, whereby the retention umbrellas ultimately positioned on the two sides of the septum eventually assume a more or less flattened form. The crosspiece centers itself automatically into the shunt to be occluded during the stretching of the umbrellas.

Because the collar protrudes past the proximal retention area of the occluder, the problem arises that the inserted implant causes embolic-related problems, in particular consecutive embolization. Because portions of the occlusion device protrudes past the septum wall and are in continuous contact with the blood, defense system reactions are also a frequent occurrence. Furthermore, a complete endothelialization of the occluder implant is often prevented.

An occlusion device made of wire braiding is additionally known from WO 95/27448 A1. This device, however, does not have a holder such that this occluder cannot be guided during introduction with an insertion instrument in the same way as is the case with the devices described above, nor can it be—in the case of a poor seating—retracted again prior to being uncoupled.

The problem therefore set out for the present invention is to refine such a braided self-centering occlusion device as known to medical technology such that the disadvantages cited above will be overcome. A particular objective is the providing of an occlusion device applicable to occluding defects of different sizes, whereby implantation of the occluder is to be a simple matter. Furthermore, the occurrence of such customary complications as dislocation, partial embolization or material fatigue to the occlusion system is to be reduced to the greatest extent possible. Above and beyond that, an occlusion device is to be provided which ensures occlusion of a septal defect with as few portions of the occlusion device as possible protruding past the septum wall so as to avoid the associated and above-cited complications.

Based on the problem as posed, it is the task of the present invention to provide an occlusion device which, in the inserted state at the proximal side of the septal defect, lies as flat as possible against the septum. The present invention moreover has the technical task of providing a method for manufacturing such an occlusion device.

These tasks are solved by the inventive occlusion device of the type specified at the outset by having the proximal retention area of the braiding exhibit a flaring toward the proximal end.

The problem relating to the process engineering of the present invention is furthermore solved by a method for manufacturing the above-cited occlusion device which is characterized by the process step of configuring a funnel-shaped hollow braiding which is bundled at a first distal end and open on an opposite second proximal end and by the process step of forming a proximal retention area at the open second end, a distal retention area at the bundled first end and interposing a cylindrical crosspiece between the proximal and the distal retention areas.

Inventive advantages are in particular seen in the provision of an intravascular occlusion device, especially for the treatment of septal defects, wherein the occluding device can be introduced into the defect to be occluded using a catheter. Because the proximal retention area of the braiding has a flaring to the proximal end of the occlusion device, a particular advantage achieved by the occlusion device is the adapting independently to the defect in the septum wall—independent of the diameter size of the defect to be occluded and independent of the thickness to the septum wall—and doing, so in such a manner that no portion of the occlusion device protrudes into the plane of the septum wall having the defect on the proximal side of the defect. The usual complications associated thereto thus no longer arise. In other words, the inserted occlusion device will be completely ingrown with the body's own tissue substantially faster than is the case with the prior art occlusion systems. By utilizing a braiding made of thin wires or threads as the starting material for the inventive occlusion device, the further advantage of long-term mechanical stability, is achieved. This largely prevents fractures from occurring in the, inserted implant's structure. The braiding furthermore exhibits sufficient rigidity. The flared contouring to the proximal end of the proximal retention area of the braiding additionally allows the proximal retention area of the device to flatten completely against the lateral edge of the defect in the inserted state and to do so virtually independently of the diameter to the defect or the thickness of the septum wall. As a result, the occlusion device can be used for a wide range of differently sized septal defects. Because there is then no need for a holder for the bundled or merging braiding at the proximal retention area, neither do any components of the occlusion device protrude past the septum wall, which prevents components of the implant from being in constant contact with the blood. This yields the advantage of there being no threat, that the body will mount defense mechanism reactions or of there being thrombembolic complications.

The inventive procedure affords the prospect of realizing a particularly simple manufacturing of the occlusion device described above. First, a funnel-shaped hollow braiding is formed, for example using a round braiding machine. The technology used here is one in which the configured braiding is bundled at the end of the length of the braiding; i.e., at what will later be the distal end of the occlusion device, while the beginning of the length of the braiding; i.e., what will later be the proximal end of the occlusion device, remains open. It is thereby possible to manufacture a funnel-shaped hollow braiding, the bundled end of which corresponds to the distal end of the finished occlusion device and the opposite open end to the proximal end of the finished occlusion device. Because a known braiding procedure is used to produce the occlusion device, the occlusion device produced exhibits mechanical properties in terms of, for example, expansion, stability, strength, etc., which can be individually adapted to the later use of the occlusion device. In advantageous manner, metallic wires or even organic threads can be worked into the braiding.

With respect to the occlusion device itself, preferred embodiments of the invention are specified in subclaims 2 to 9 and, with respect to the manufacturing process, in subclaims 11 to 14.

It is thus preferably provided for the occlusion device to have the braiding consist of nitinol or of another shape-memory material or material having memory effect. Such other material could conceivably be, for example, copper-zinc-aluminum alloys, gold-cadmium alloys or even ferrous alloys such as e.g. iron-manganese-silicon alloys, or also plastics, all which are characterized by their extremely high memory capacity. It is particularly provided for the braiding of the inventive occlusion device to be formed from a shape-memory polymer based on, for example, polyanhydride matrices or on polyhydroxycarboxylic acids. These are, synthetic, biodegradable materials which have a thermally-induced shape-memory effect. Yet also conceivable would be other shape-memory polymers such as, for example, block copolymers as described for example in the special edition of Angewandte Chemie¹ 2002, 114, pages 2138 to 2162, by A. Lendlein and S. Kelch. By making use of such a material, it is possible to utilize a funnel-shaped hollow braiding for the starting body of the occlusion device, created for example by means of a round braiding method, which is open at its leading end and bundled at its trailing end. Said starting body is then subsequently brought into the desired form for the occlusion device by means of a molding and heat treatment procedure. Other treatment procedures are of course also conceivable here.
¹“Applied Chemistry”

It is particularly preferred to have the braiding, after it has been given a suitable form by means of the molding and heat treatment procedure, taper to the diameter of one of the catheters used in the intravascular surgical procedure. This thus makes it possible to introduce the occlusion device for occluding the defect by inserting the catheter into a vein so that an operation in the literal sense is no longer necessary. If the braiding is made of nitinol, for example, or of another material having shape-memory or memory effect properties, the occlusion device tapered to the diameter of the catheter is that of a “self-expanding device,” which unfolds by itself after exiting the catheter such that the two retention areas can accordingly position on the proximal/distal sides of the defect. The design to the contiguous braiding of the inventive occlusion device moreover occasions an occlusion device which is a self-expanding and self-positioning occlusion system and which prevents permanent mechanical stresses from occurring between the inserted occlusion device and the septum wall.

Provided as a conceivable realization is that the proximal retention area of the braiding exhibits a tulip-shaped flared contouring,toward the proximal end.

It would furthermore be conceivable for the proximal retention area to exhibit a bell-shaped contouring flared to the proximal end. This would thus allow the occlusion device to be used in the treatment of various different defects, in particular ventricular septal defects (VSD), atrioseptal defects (ASD) as well as persistent ductus arteriosus Botalli (PDA), whereby an optimized contouring to the proximal retention area can in principle be selected for a plurality of defects of differing sizes and types. Of course, other contourings are also conceivable here.

Particularly preferred is for the occlusion device to exhibit at least one fabric insert arranged within the crosspiece or at the proximal retention area of the occlusion device. This fabric insert serves to close any remaining gaps in the crosspiece and the expanding diameters of the occlusion device after the device has been inserted into the defect. The fabric insert is for example stretched over the open end of the braiding in such a manner that it can cover the opening like a cloth. The advantage to this design lies in the fact that the lateral edge of the proximal retention area is flush with the septum and less foreign material is introduced into the body of the patient. The fabric inserts can, for example, be made of Dacron. Of course other materials and other positionings to the fabric insert in or on the occlusion device are also conceivable here.

With respect to the method, it is preferably provided for the process step of forming the retention area and the crosspiece to include a molding and heat treatment procedural step. This is of particular advantage when the configured, funnel-shaped hollow braiding is made of nitinol or of another material, especially polymer, which has shape-memory properties or effect. Preferably provided for the inventive occlusion device is forming the braiding from a shape-memory polymer which is based, for example, on polyanhydride matrices or on polyhydroxycarboxylic acids. These are synthetic, biodegradable materials which have a thermally-induced shape-memory effect. Yet also conceivable would be other shape-memory polymers such as, for example, block copolymers as described for example in the special edition of Angewandte Chemie 2002, 114, pages 2138 to 2162, by A. Lendlein and S. Kelch. It is a simple matter to bring such materials into the applicable final form using a combination of molding and heat treatment procedural steps. A final formed occluder can then be tapered to the dimensions of a catheter, for example. After exiting the catheter, the occlusion device then unfolds by itself and again assumes that profile to the funnel-shaped hollow braiding to which the occlusion device was molded during the manufacturing process by means of the molding and heat treatment step.

It is preferred for the funnel-shaped hollow braiding to be manufactured in such a manner that the thin wires or threads constituting the finished braiding intertwine at the proximal end of said braiding when forming the funnel-shaped hollow braiding. This represents a conceivable and readily realizable manner of producing an occlusion device in accordance with the present invention, the proximal retention area of which exhibits a form flared to the proximal end. Of course, other manufacturing methods are naturally also conceivable.

The following will make reference to the drawings in providing a more precise detailing of preferred embodiments of the inventive occlusion device as well as of a round braiding machine to provide clarification by example of the inventive manufacturing process for the occlusion device.

Shown are:

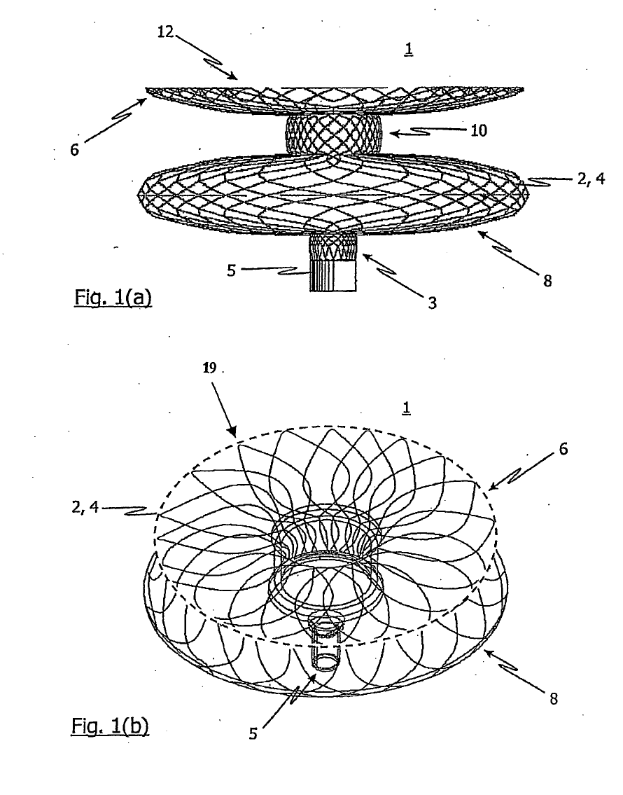

FIG. 1(a) a schematic side view of a preferred first embodiment of an occlusion device according to the present invention in expanded state;

FIG. 1(b) a perspective view of the FIG. 1(a) first embodiment of the occlusion device in expanded state;

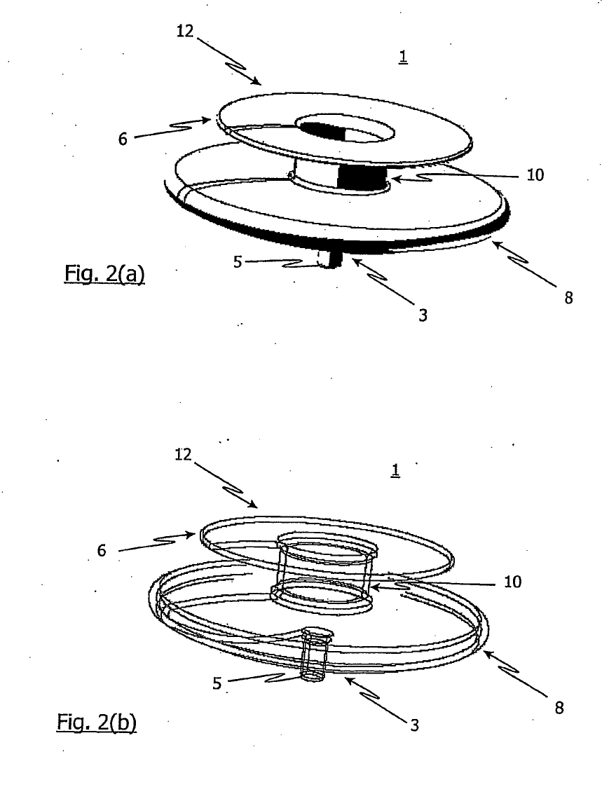

FIG. 2(a) a perspective view of a preferred second embodiment of an occlusion device according to the present invention in expanded state;

FIG. 2(b) a contour-only representation of the FIG. 2(a) second embodiment of the occlusion device;

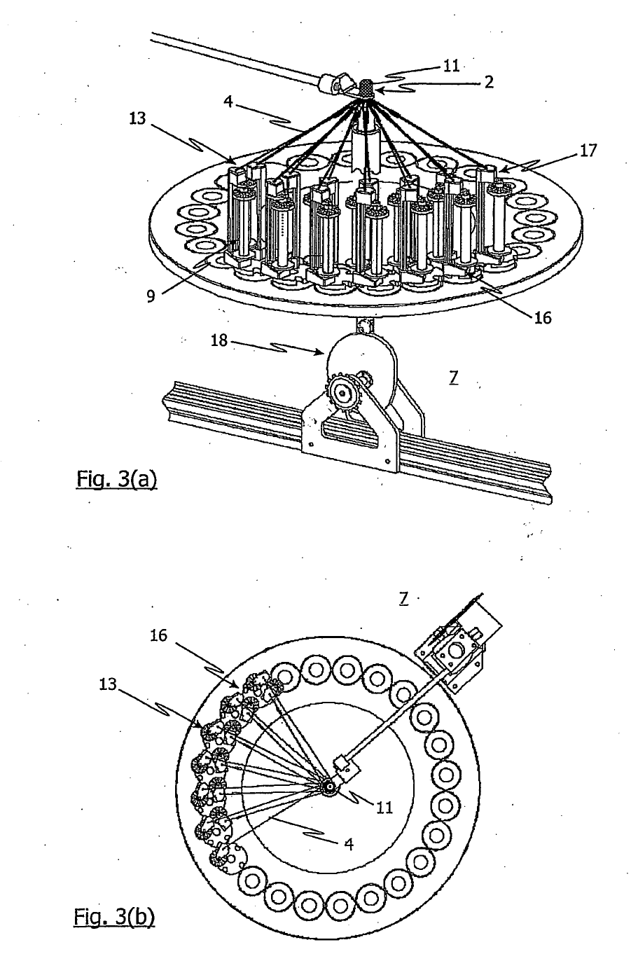

FIG. 3(a) a three-dimensional view of a round braiding machine for illustrating the inventive manufacturing method for the occlusion device;

FIG. 3(b) a top plan view onto the round braiding machine depicted in FIG. 3(a)for illustrating the inventive manufacturing method for the occlusion device;

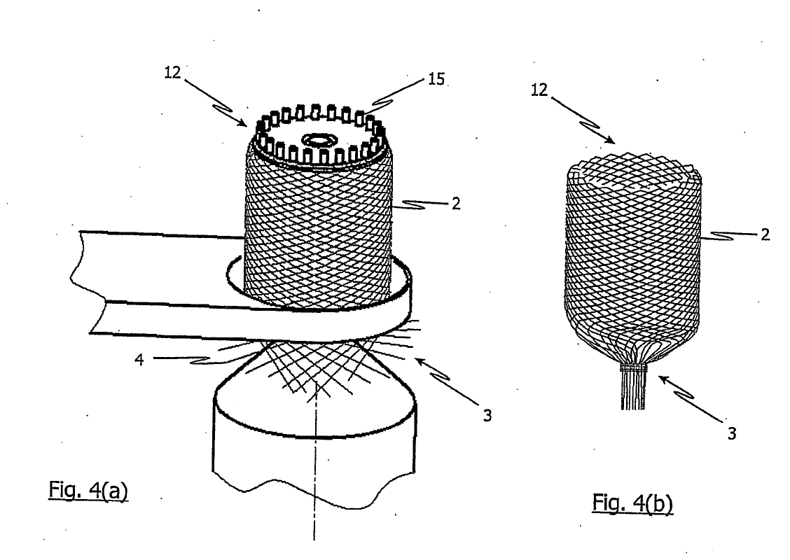

FIG. 4(a) a detail view of the braiding head of the round braiding machine depicted in FIG. 3; and

FIG. 4(b) a braiding manufactured with the braiding head shown in FIG. 4(a) which serves as the starting structure for the inventive occlusion device.

FIG. 1(a) is a schematic side view of a preferred first embodiment of an occlusion device 1 according to the present invention in expanded state and FIG. 1(b) shows a perspective view of the first embodiment of the occlusion device 1 shown in FIG. 1(a). Occlusion device 1 essentially consists of a braiding 2 of thin wires or threads 4, preferably made from nitinol or from another material having shape-memory properties or effect. Braiding 2 exhibits sufficient flexibility such that occlusion device 1 can be tapered to the diameter of a catheter used in an intravascular surgical procedure (explicitly not shown). Due to the material's memory effect, the occlusion device 1 tapered as such has a shape-memory function such that device 1 self-expands after exiting the catheter and reassumes the pre-defined form which corresponds to its use. This usually transpires after occlusion device 1, initially arranged in the catheter, is positioned at the location to be treated.

As depicted in FIGS. 1(a) and 1(b), occlusion device 1 in the expanded state exhibits a proximal retention area 6, a distal retention area 8 and a cylindrical crosspiece 10 arranged between said proximal and distal retention areas 6, 8. The two retention areas 6, 8 serve to occlude a defect and/or shunt located in a septum. This ensues by said areas 6, 8 positioning against the two sides of the shunt to be occluded while crosspiece 10 transverses the shunt. Inventive occlusion device 1 thus represents an occlusion system which is introduced into the body of a patient and positioned at the location for which it is intended by means of a minimally invasive procedure; i.e., using a catheter and guidewires, for example.

The design to inventive device 1 is thus based on the principle of having occlusion device 1 be tapered to the dimensions of the catheter. After exiting the catheter, retention areas 6, 8 then unfold by themselves and position themselves on both sides of the septum. The inventive design furthermore provides for occlusion device 1 to be that of a self-positioning and self-centering system. Crosspiece 10 thereby has the length of the septum/atrial septum wall in order to ensure a solid deployment of retention areas 6,8 at the septum wall.

In contrast to the conventional occlusion systems known from the prior art, in which a self-expanding umbrella serves as proximal retention area 6, the proximal retention area 6 of the present invention has a bell or tulip-shape flare to proximal end 12 so that no material of the implanted occlusion device 1 whatsoever can extend past the septum wall in the proximal area of the patient's organ. The flared contouring to proximal end 12 of proximal retention area 6 further ensures that the edge of proximal retention area 6 always lies flush with the septum wall. This occurs over a relatively wide area independent of the diameter of the defect or the thickness of the septum/atrial partition and allows for a complete endothelialization to be realized relatively quickly subsequent implantation of occlusion device 1 and the patient's body mounts no defense mechanism reactions since the blood is effectively prevented from coming into contact with the material of implant 1.

Because of the self-expanding property to implant 1 based on the memory effect of the materials used, inventive occlusion device 1 exhibits a self-centering function in the shunt, in the defect of the septum respectively. Furthermore, occlusion device 1 can be withdrawn at any time up to the uncoupling of the guidewires of the insertion instrument.

Inventive occlusion device 1 furthermore comprises fabric inserts, which are explicitly not shown in the present drawings. These fabric inserts consist mostly of Dacron material. It is hereby conceivable to incorporate the fabric inserts within the interior of crosspiece 10 or at the proximal end 12 of retention area 6 in order to be able to completely occlude the defect/shunt in the septum wall. The fabric inserts can be incorporated by, for example, bracing same within occlusion device 1. The implant 1 inserted into the body will then be completely ingrown by the body's own tissue within just a few weeks or months.

The braiding 2 serving as the base structure for inventive occlusion device 1 exhibits sufficient rigidity to clamp the fabric insert and have it remain in its position.

Braiding 2 is centralized into a holder 5 at the distal end 3 of distal retention area 8. This is hereby realized in that an internal threading can be manufactured within holder 5 which then serves to engage with a guidewire of a not-shown insertion instrument while occlusion device 1 is being introduced to the applicable position relative the location of the defect in the septum, for example, by means of an intravascular surgical procedure.

After occlusion device 1 has been positioned in the shunt/defect, the engagement between the guidewire of the insertion instrument and distal end 3 is disengaged. Of course, it is also conceivable to apply a differently-configured device in place of an internal threading in holder 5 of distal end 3.

FIG. 2(a) is a schematic three-dimensional view of a preferred second embodiment of the occlusion device 1 according to the present invention in expanded state and FIG. 2(b) is a representation of the FIG. 2(a) view of the second embodiment of the occlusion device 1 showing only the contours for simplification purposes. To allow even further simplification,sa detailed representation of the braiding 2 serving as the base structure is not shown, and the contouring to occlusion device 1 is depicted as a closed surface. Said occlusion device 1 exhibits a flatter proximal retention area 6 compared to that of the first embodiment. Depending upon intended application, proximal retention area 6 is configured in a more or less distinctly tulip shape. Yet it would also be conceivable here to have a bell-shape tapering to proximal end 12 or a contouring which is almost saucer-shaped.

FIG. 3(a) depicts a round braiding machine 7 in order to illustrate the manufacturing process for occlusion device 1 according to the invention, and FIG. 3(b) shows a top plan view of the round braiding machine 7 depicted in FIG. 3(a). In contrast to the known braiding methods, where all the threads or wires 4 are gathered into one bundle and stretched to an extractor device at the leading end of braiding 2, in the inventive method, the material supply is stretched from every second spool 9 to a braiding head 11 and from there to each respective next spool 13 or a multiple of its center distance. Those spools 13 without a material supply contain only an auxiliary thread which reaches up to at least braiding head 11. The end of the material supply is connected to the end of the auxiliary thread as close as possible to the auxiliary thread spool by bolt 14.

Braiding head 11, shown in detail in the figure, has a crown-like shape and is provided with form elements 15 which enable threads or wires 4 to be hooked. Form elements 15 can be lowered in order to be able to be hooked and unhooked. Braiding head 11 is axially positioned at the center of the orbit of impellers 16 such that the threads or wires 4 are aligned at a flat downward angle to bobbin 17 of braiding machine 7.

After all the wires 4 needed for braiding 2 are joined and tightened, the braiding 4 commences in customary fashion, as impellers 16 rotate around the center while bobbins 17 shift from impeller to impeller, their orbits thereby crossing. The infeed for braiding 2 is realized via a cam plate 18 based on the revolutions of impellers 16. The length to the braiding which can be manufactured with this method is proportional to the circumference and pitch of braiding 2 as well as to the length of the end of the wire or thread connected to the auxiliary thread. Subsequent the braiding, the free ends are bundled or gathered, cut from the material supply and uncoupled from the auxiliary thread. The funnel-shaped hollow braiding 2 thus produced is open at its leading end and bundled at its trailing end. The wire bundle is devised such that an internal threading can be produced therein for engaging with the guidewire of an insertion instrument.

In the subsequent material-dependent molding and heat treatment procedure, braiding 2 is brought into the desired form of occlusion device 1. The starting structure is suitable for producing occlusion device 1 for the treatment of ventricular septal defects (VSD), atrioseptal defects (ASD), as well as persistent ductus arteriosus Botalli (PDA).

As seen from the perspective of holder 5 and depending upon configuration, an expanded diameter (i.e., distal retention area 8) is formed, followed by crosspiece 10, to which another expanded tulip-shaped diameter (i.e., proximal retention area 6) is joined. Another embodiment exhibits a bell shape open to the top.

Since braiding 2, which serves as the base for occlusion device 1, cannot as such fully occlude a defect, fabric inserts are introduced into crosspiece 10 and in the expanding diameters—the distal and/or proximal retention areas 6, 8. Said fabric inserts, preferably consisting of Dacron, then close the gaps remaining in braiding 2 in the inserted state of occlusion device 1. Said fabric inserts are preferably secured such that they can be stretched over the proximal opening like a cloth.

FIG. 4(a) shows a detailed view, of braiding head 11 of the round braiding machine 7 from the FIG. 3 representation and FIG. 4(b) shows a braiding 2,produced with the braiding head 11 as shown in FIG. 4(a), which serves as the starting structure for inventive occlusion device 1. It can clearly be seen here that braiding 2 serving as the base structure for occlusion device 1 is configured as a tubular braiding 2 open to its top which only needs to be provided with a holder 5 at its end 3, while the threads or wires 4 at the opposite side 12 are intertwined virtually from the center thereof.

List of Reference Numerals

| | |
|---|---|
| 1 | occlusion device |
| 2 | braiding |
| 3 | distal end |
| 4 | thread, wire |
| 5 | holder |
| 6 | proximal retention area |
| 7 | braiding machine |
| 8 | distal retention area |
| 9 | spool |
| 10 | crosspiece |
| 11 | braiding head |
| 12 | proximal end |
| 13 | spool |
| 14 | bolt |
| 15 | form element |
| 16 | impeller |
| 17 | bobbin |
| 18 | cam plate |
| 19 | outer edge |

1. An occlusion device consisting of a braiding of thin wires or threads given a suitable form by means of a molding and heat treatment procedure, having a proximal retention area and a distal retention area, whereby the ends of the wires or threads converge into a holder in the distal retention area, and having a cylindrical crosspiece interposed between said proximal and distal retention areas, whereby the two retention areas are positioned on the two sides of a shunt to be occluded in a septum by means of an intravascular surgical procedure while a crosspiece transverses the shunt,
wherein a proximal retention area of the braiding exhibits a flaring toward a proximal end.

2. An occlusion device in accordance with claim 1, wherein the braiding is composed of nitinol or of another shape-memory material.

3. An occlusion device in accordance with claim 2, wherein the braiding is formed from a shape-memory polymer, preferably based on a polyanhydride matrix or on polyhydroxycarboxylic acids.

4. An occlusion device in accordance with claim 2, wherein the braiding is formed from a shape-memory polymer of a block copolymer form.

5. An occlusion device in accordance with claim 1, wherein the braiding tapers to a diameter of one of a plurality of catheters used in the intravascular surgical procedure.

6. An occlusion device in accordance with claim 1, wherein a proximal retention area of braiding exhibits a flattened tulip-shaped flared contouring to the proximal end.

7. An occlusion device in accordance with claim 1, wherein the proximal retention area of the braiding exhibits a bell-shaped flared contouring to the proximal end.

8. An occlusion device in accordance with claim 1, wherein the wires or threads of the braiding at the open end of the proximal retention area are looped back to the closed end of the distal retention area and secured there in a holder.

9. An occlusion device in accordance with claim 1, wherein at least one fabric insert is arranged in crosspiece or in the proximal retention area for complete occluding of the shunt.

10. A method of manufacturing an occlusion device comprising:
a) configuring a funnel-shaped hollow braiding, whereby said hollow braiding is bundled at a first distal end and remains open on an opposite second proximal end; and
b) forming a proximal retention area at the open second end, a distal retention area at the bundled first end, and interposing a cylindrical crosspiece between said proximal and said distal retention areas.

11. The method in accordance with claim 10, further comprising configuring a holder at the bundled distal end of said funnel-shaped hollow braiding.

12. The method in accordance with claim 10, wherein the wires and threads of the braiding at the outer edge of the-flattened tulip shape of the open end of the proximal retention area are looped back to the closed end of the distal retention area and are bundled and secured there in the holder.

13. The method in accordance with claim 10, wherein the step of forming retention areas and crosspiece includes a molding and/or heat treatment.

14. The method in accordance with claim 10, wherein a funnel-shaped hollow braiding structure is produced such that the thin wires or threads that constitute finished braiding are intertwined at the proximal open end of the braiding when the the funnel-shaped hollow braiding is formed.
